(19) United States
(12) Patent Application Publication
(10) Pub. No.: US 2017/0156800 A1
(43) Pub. Date: Jun. 8, 2017

(54) Surgical Targeting Systems and Methods

(71) Applicant: Roy Anthony Brown, Carlsbad, CA (US)

(72) Inventor: Roy Anthony Brown, Carlsbad, CA (US)

(21) Appl. No.: 14/659,497

(22) Filed: Mar. 16, 2015

(51) Int. Cl. A61B 34/20; A61B 90/13; A61B 90/00; A61B 6/02

(52) U.S. Cl. CPC A61B 34/20; A61B 6/02; A61B 90/13; A61B 90/39; A61B 2034/2051; A61B 2090/3983

(57) ABSTRACT

The present invention relates to a system and method to aid the placement of surgical devices under radiographic image guidance. More particularly, embodiments of the invention relate to a system utilizing radiopaque markers, an external light source projected onto the skin or surgical site in conjunction with a target guide holder. Using a radiographic image to identify landmarks for skin entry and bone entry points to facilitate the accurate placement of surgical devices. An exemplary system utilizes a radiopaque marker, target guide holder and external laser markers to determine intra-operative angles, trajectories and positioning coordinates to facilitate placement of needles, guide wires, surgical hardware, trocars and cannulae for the surgical placement of orthopedic implantation devices.

### CROSS-REFERENCE TO RELATED APPLICATIONS

Provisional Application Ser. No. 61/954,250, filed on Mar. 17, 2014.

### BACKGROUND OF THE INVENTION

Field of the Invention

The present invention relates to a system and method to aid the placement of surgical devices under radiographic image guidance. More particularly, embodiments of the invention relate to a system utilizing radiopaque markers, an external light source and targets. Light is projected onto the skin or surgical site over a target in conjunction with a radiographic line marker superimposed on a fluoroscopic image to identify bone landmarks and angles so that skin entry points can be identified. This can be augmented by the use of a target system that is held in place by a bedside rail mounted mechanical arm that can hold any position desired. This allows rigid guidance of guide wire to facilitate the accurate placement of surgical implant or devices. An exemplary system utilizes a radiopaque marker, external laser markers and a target to determine intra-operative angles, trajectories and positioning coordinates to facilitate placement of needles, guide wires, trocars and cannulae for the surgical placement of orthopedic implantation devices.

Description of the Related Art

Many fluoroscopy systems on the market possess a laser “aimer” or pointer that is used in conjunction with the imaging source. One example is the Smart Laser Aimer from GE OEC (GE Healthcare, Salt Lake City, Utah). The laser pointer is mounted on the Image Intensifier of the C-arm and is used as a line of sight pointer. The laser light illuminates the center point on the surgical site where the x-ray beam will image if activated, giving the user a more accurate location of the image. It does not accurately place the image in global 3-dimensional space, nor does it provide an accurate location with respect to anatomical landmarks. The user must rely on more complex image guidance systems intra-operatively, or 3-D image reconstruction software pre-operatively in order to obtain more accurate information for precise instrument placement. One example of intra-operative guidance systems is the Stealth Station from Medtronic. Such systems require a dedicated piece of equipment to transmit and receive signals and markers on the surgical instruments to track the position and orientation of each instrument. Dedicated software and image storage are also required to incorporate guidance system information into preoperative or intraoperative images. Such systems do not have the benefit of the present invention of being compatible with any commercially available imaging equipment and surgical instruments.

There are many targeting or aiming apparatus for making bores in bones in register with bores has been disclosed in the targeting system previously disclosed in U.S. Pat. No. 5,031,2013 that utilized a laser and a fixed target in combination with x-rays. More recently, there have been articles focusing on targeting with a complex computer aided such as, “Percutaneous Lumbar Pedicle Screw Placement Aided by Computer-Assisted Fluoroscopy-based Navigation” by Benson P. Yang, MD, Melvin Wahl, MD, CARY S. Idler MD, Spine:37(24):2055-2060. There has also been other publications such as, “Accuracy of Fluoroscopically assisted laser targeting of the cadaveric thoracic and lumbar spine to place transpedicular screws” by Schwend, R M, Dewire P J, Kowalski T M; J Spinal Disord. 2000 October; 13 (5): 412-8; “Pedicle Guide for Thoracic Pedicle Screw Placement” by Kingsley O. Abode-Iyamah MD; Luke Stemper BS; Shane Rachman BS; Kelly Schneider BS; Kathryn Sick BS Patrick W. Hilton MD, University of Iowa Hospitals and Clinics; and the work of C. Grady McBride at the Orlando Orthopaedic Center, where reduction of fluoroscope times resulted in the use of a targeting device in parallel for insertion of a guide wire. In none of these pieces of prior art are there the safety, ease of use, and the efficiency of the instant invention.

The fluoroscopy systems operate on either a continuous or pulsing system for x-rays to permit continuous or near continuous monitoring of the medical procedure involved. In either situation there is still a need to reduce or limit the exposure of patients to the exposure of the x-ray radiation. Timing is critical, but in the surgeries utilizing today's fluoroscopy systems there is somewhat a hit and miss approach to finding the landmarks need for the attachment of screws for spinal surgery, as the procedure follows a general methodology of measurement and a grid pattern that often does not consider the thickness of a patient's soft tissue and muscle from the area of attachment such as the pedicles of the spine. The use of Jamshidi needles, trocars and cannulae for certain surgeries help limit wound size and openings, but the degree of precision desired is still not met using the current methods, even with complex software and robotics. The degree of precision has greatly improved, but the accuracy of the puncture for attaching screws in the body still relies on an estimate of the location of the incision without an exemplar or marker to follow or a more accurate place in which to make the incision. For example, in spine surgery the standardized methodology will be to measure from the midline to a fixed distance to make an incision with limited regard to the angle of entry and if the landmark is not hit on the first attempt there are continued attempts and the need for dealing with tissue and muscle as the trocar or cannula is being positioned to find the pedicle landmark. This increases unnecessary exposure to x-rays and the increased chance of injury to tissue and muscle.

Also, the focus is minimally invasive surgery is to limit the need for opening the body and increase the risk of infection and healing. In the use of robotics, for instance, section of the spine still need to be exposed to attach the rail for the robotic system to be used during spine surgery. While this may be an improvement over opening the entire area of the spine, it still creates issues around infection and healing of the wounds. While the methodologies used to get towards minimally invasive surgery have improved there is significant opportunity for an increase in accuracy to go along with the increase in precision.

### SUMMARY OF THE INVENTION

The present invention is a system and method used in conjunction with fluoroscopic imaging systems to identify bone landmarks and angles, skin entry points and trajectories and a target guide holder in order to aid the placement of surgical instruments, such as guide pins, needles, trocars, fixation hardware and cannulae. The system's utility is not limited to a particular anatomical location, and thus can be used in a wide range of variety of surgical applications. In addition to the spine surgery application detailed below, it can be used in human, veterinary, or training models for cranial, hip, knee, and wrist surgery, for example.

The system comprises of an adjustable radiopaque bar marker mounted below external light sources, such as visible light sources or lasers, the associated mounting hardware on the imaging system and a separate targeting guide holder. The mounting hardware allows the radiopaque marker to translate around and across the circumference and face 360° around the image intensifier. Limiting the radiopaque bar/visible light marker to pivot on its axis to −+5° insures projected lines under fluoroscopy stay within the limits of beam divergence parameters for accuracy of visible light on patients skin. The system is used in conjunction with commonly available pre-operative images and commercially available intra-operative radiography equipment. A pre-operative image of the intended surgical site is taken using computed tomography (CT) or magnetic resonance Imaging (MRI). On this image, the anatomy of the intended surgical site is seen and used to pre-operatively plan the angles, trajectories and positioning of the surgical instruments by superimposing points and lines on the pre-operative image. From this pre-operative plan, the intended entry point on the skin and angulation of each instrument is planned. Once skin entry point is established the Target guide holder is placed in the surgical field. Using a 2-axis inclometer the AP angle can be applied in the x plane. While in the lateral plane, the target guide holder is aligned with the light line and the y angle can be read off the inclometer. The pre-operative planning step may be performed manually on a printed image or electronically using commercially available software and a digital image. Additional lines are constructed on the pre-operative image by projecting the position of the intended entry points on the skin in the orthogonal planes to be used for intra-operative imaging at the time of surgery. The intersection of the orthogonal projection lines with anatomical landmarks indicates which anatomical landmark to use in intra-operative imaging to align the system. Intra-operative planning may also be performed in the same manner using intra-operative images.

Intra-operatively, the light source is mounted to a commercially available radiographic imaging system, such as a fluoroscope or portable x-ray. The light beams are projected as a line onto the skin at the surgical site. The radiopaque bar markers and light sources are located in known positions with respect to the imaging system. The radiopaque bar markers are imaged with the anatomical location of interest, and the light sources are projected onto the skin in the plane of the intended entry point determined in pre- or intra-operative planning. The intersection of two linear light beams in orthogonal planes, typically but not necessarily the anterior/posterior (AP) and medial/lateral (ML) planes, clearly mark the entry point of the surgical instruments on the skin of the patient. The orientation of the surgical instruments at the entry point is set using the target guide holder, an angularly adjustable, bi-planar, mechanical guide to set the angle of the instruments in both orthogonal planes per the pre- or intra-operative plan. In some instances a phenomenon known as beam divergence becomes a factor as the area of interest moves away from the center of the image. The nature of the design in the adjustable bar marker allows for visual conformation on the intra-operative radiograph that the bar is in alignment with the divergence. Therefore the light beam on the skin is in true alignment. The system thereby provides accurate both the positioning coordinates and the orientation of the surgical instrument to the surgeon, such that if the resulting trajectory is followed, the instrument will reach the intended internal surgical site without direct visualization by dissection or repeated radiographic exposures.

An example of the method using the present invention and a pre-operative plan includes an axial pre-operative image, also known as a “slice”, of the intended surgical site is taken using computed tomography (CT) or magnetic resonance imaging (MRI). On this image, the anatomy of the intended surgical site is seen in cross-sectional axial view (a view not commonly available intra-operatively) and used to pre-operatively plan the angles, trajectories and landmark positioning of the surgical instruments. From this pre-operative plan, the intended skin entry point is defined for the AP plane. An example of the method using the present invention and an intra-operative plan includes a lateral intra-operative image using fluoroscopy or portable x-ray. On this image, the anatomy of the intended surgical site is seen in side elevation and used to plan the angles, trajectories and positioning of the surgical instruments. From this intra-operative plan, the intended skin entry and bone entry point is defined in the ML plane. When the two exemplary methods are used together, for example in spinal surgery, the intersection of the AP and ML planes using the light beam mark the surgical skin entry point. The use of the target guide holder insures no human initiated deviation from plotted trajectory is introduced during insertion. This method and device are ideal for minimally invasive procedures including but not limited to discectomy, pedicle screw placement for fixation, facet fusion, facet joint injection, nerve ablation, vertebroplasty.

Another example of the method is for training surgeons in using the invention for improved performance and accuracy. The intersection of the AP and ML Planes using the light beam mark the surgical skin entry point and the surgeon get use to understanding the various degrees of entry required, such that in the case of the back surgery of the previous paragraph, the angles become familiar to the surgeon through training and they become more accurate in the surgical entry point and the angles of that entry point. Clearly, the invention is applicable for use with not only spinal surgery but also orthopedic surgeries involving shoulder, hips, joints, wrist, arms, legs, ankles hands and feet.

### BRIEF DESCRIPTION OF THE DRAWINGS

In order to better understand the invention and to see how it may be carried out in practice, some preferred embodiments are next described, by way of non-limiting examples only, with reference to the accompanying drawings, in which like reference characters denote corresponding features consistently throughout similar embodiments in the attached drawings.

FIG. 1 is a translucent illustration of the lateral and posterior views of the surgical patient, with the linear light beam externally positioned in the posterior view and the intended trajectory of a surgical instrument through the body in the lateral view.

FIG. 2 is an example of the instrument trajectory of FIG. 1A as projected on a radiographic image.

FIG. 3 is an illustration of the determination of the surgical angle and projection of the entry point of the skin on an anatomical landmark on a pre-operative CT image.

FIG. 4 is an illustration of completing the A/P positioning technique by locating the anatomical landmark.

FIG. 5 is radiograph example of the technique of FIG. 5.

FIG. 6 is an illustration of the guide pin insertion.

FIG. 7 is radiograph example of the technique of FIG. 7

FIG. 8 is an illustration of final positioning of the guide pin.

FIG. 9 is an illustration of the lateral and posterior views of the cranium, externally positioned in the posterior view and the intended trajectory of a surgical instrument through the skull in the lateral view. Straight vertical and horizontal lines illustrate radiopaque markers and contoured lines illustrate skin incision trajectories.

FIG. 10 is an illustration of a Fluoroscopic system in a side view.

FIG. 11 A is a perspective view of a Fluoroscopic C-Arm System.

FIG. 11 B is a side view of a GE Fluoroscopic C-Arm System.

FIG. 12 is a perspective view of the collar for the image intensifier with the light source and the radiopaque marker.

FIG. 13 is a perspective view of the light source and radiopaque marker and how the light source and holder have movement laterally.

FIG. 14 is a side view of the collar for the image intensifier with the light source and radiopaque marker.

FIG. 15 is a front view of the collar for the image intensifier with the light source and radiopaque marker.

FIG. 16 is a top view of the collar for the image intensifier with the light source and radiopaque marker.

FIG. 17 shows perspective views of an alternative the collar for the image intensifier where the face rotates around the image intensifier.

FIG. 18 is illustrative of the pre-surgical preparation.

FIG. 19 illustrates the view from the monitor of the fluoroscope of the of the radiopaque marker.

FIG. 20 illustrates an alternative collar for the image intensifier.

FIG. 21 A Illustrates a Jamshidi, a stylet and a target guide holder.

FIG. 21 B illustrates an inclometer for determine the AP angle and the lateral angle.

FIG. 22 illustrates a hand and wrist having a plate with screws.

FIG. 23 illustrates a Humeral Shaft with a plate and screws.

### DETAILED DESCRIPTION OF THE PREFERRED EMBODIMENTS

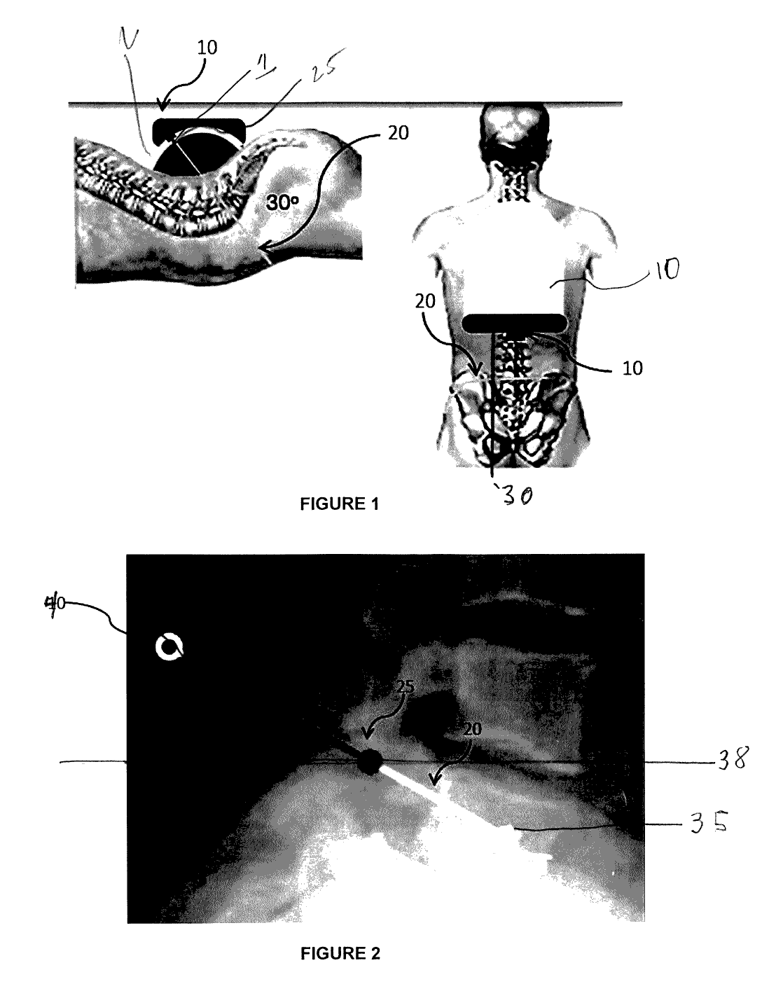

First, the light source 1 in FIG. 1A must be positioned. A collar system 2 will fit the image intensifier 10 incorporating the light source 1 and the radiopaque marker 25. As shown in FIG. 1A and FIG. 1B, using the radiopaque marker 25 on the face of the laterally positioned image intensifier 10 fluoroscopically the light source trajectory 20 is determined through the spine segment. By superimposing the marker over the anatomy, the system automatically places the laser marker over the skin as shown at 30. This determines both the angle and latitude position on the skin to start the procedure.

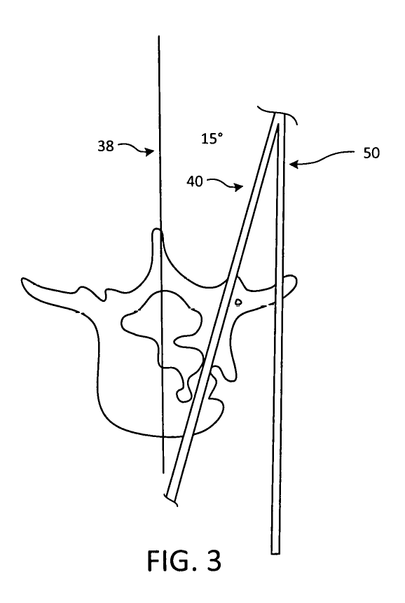

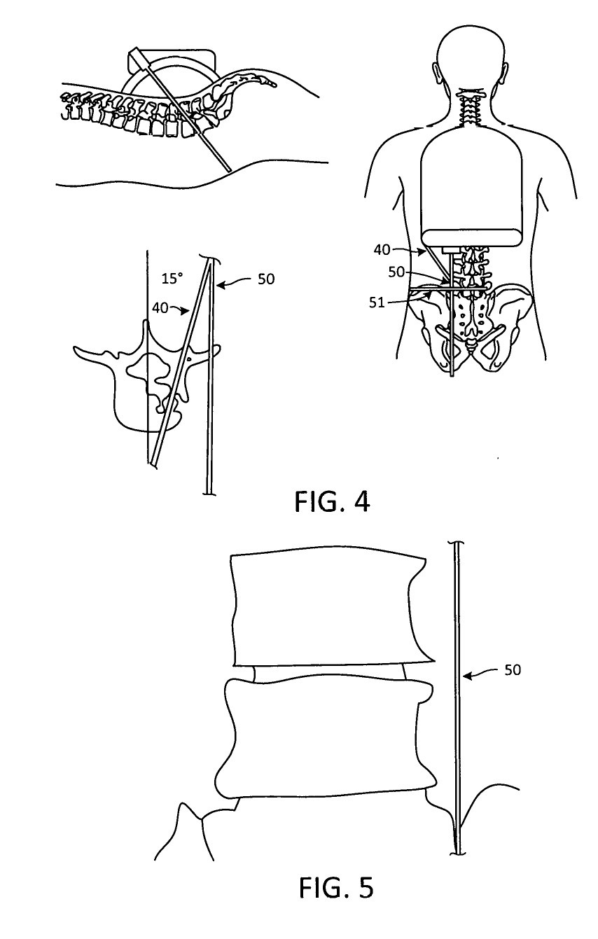

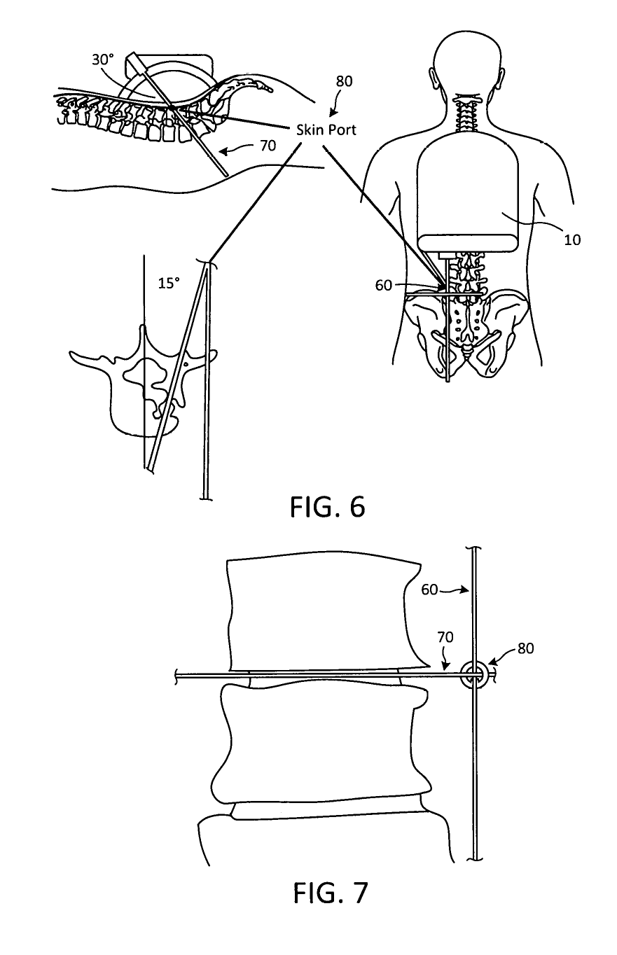

Next, the A/P position must be determined by looking at the preoperative axial view of the target in question. In FIG. 2, the target in question is a vertebral body 35. The midline 38 is determined, an azimuth through the pedicle or structures desired is positioned, and an angle is determined that would effectively produce the correct trajectory 40 through the anatomy. For example, FIG. 3 illustrates an angle of 15 degrees at the feature of interest, the end of transverse process. The A/P landmark is determined by using the axial view (FIG. 4) by looking down through the anatomy from the point in which the azimuth exits the body posteriorly 40. FIG. 4 illustrates the example of the trajectory overlying the end of the transverse process 50. The intersection of 50 and 40 is shown at 51. Finally, using the radiopaque marker on the face of the A/P intensifier, the marker is fluoroscopically superimposed over the landmark previously identified. In the example shown in FIG. 4, FIG. 5, and FIG. 6 this is the end of the transverse process.

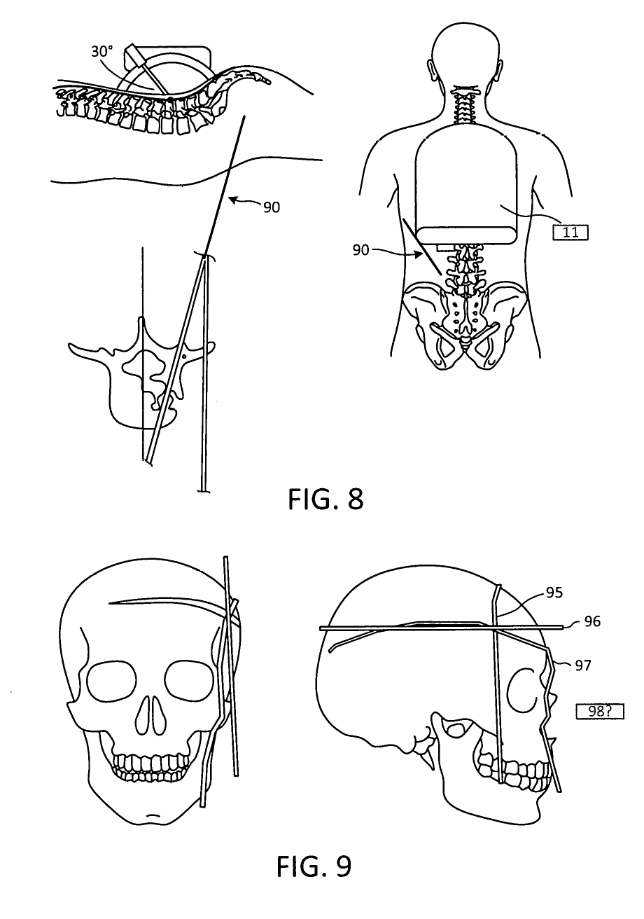

The inclometer guide pin 90 can now be deployed. Using both laser beam 60 and laser beam 70 as reference lines on the skin, the skin port or entry point 80 is established as illustrated in FIG. 7. Next, the inclometer guide pin is positioned into the target holder and with the aid of the positioning arm positioned at pre-established angle in AP and target guide holder ML centerline brought into alignment with lateral laser light beam. In the example of FIG. 8, the angles are shown as 30 degrees lateral, 15 degrees A/P. Then the inclometer guide pin is replaced with the procedural guide pin then advanced to its fully inserted position as shown in FIG. 8. Once the guide pin is successfully inserted, the procedure can begin.

FIG. 9 is an illustration of the lateral and posterior views of the cranium, externally positioned in the posterior view and the intended trajectory of a surgical instrument through the skull in the lateral view. Straight vertical 95 and horizontal 96 lines illustrate radiopaque markers and contoured lines 97 and 98 illustrate skin incision trajectories.

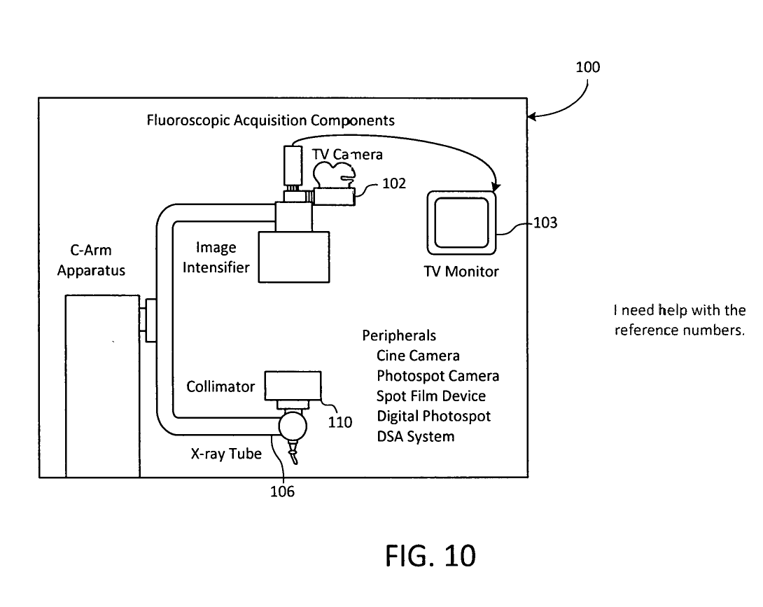

FIG. 10 shows a representation of the side of view of a fluoroscope system 100 having an image intensifier 101, a CCD camera 102, a monitor 103, a C-Arm 104, a collimator 105 and an X-ray tube 106. The fluoroscope system 100 is known as a C-Arm system. The directed x-ray radiation generated by the X-ray tube 106 passes through the body part at position between the collimator 105 and the image intensifier 101 that is transmitted via the CCD camera 102 to the monitor 103. The X-rays are either continuous or pulsing so that the surgeon can view the surgery via the monitor 103 in real time.

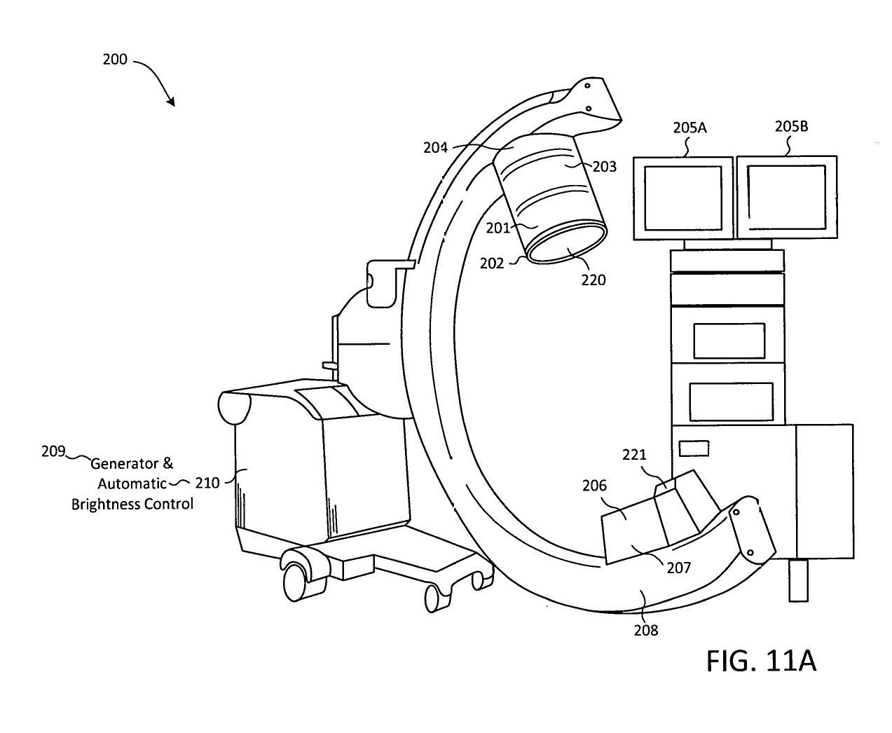

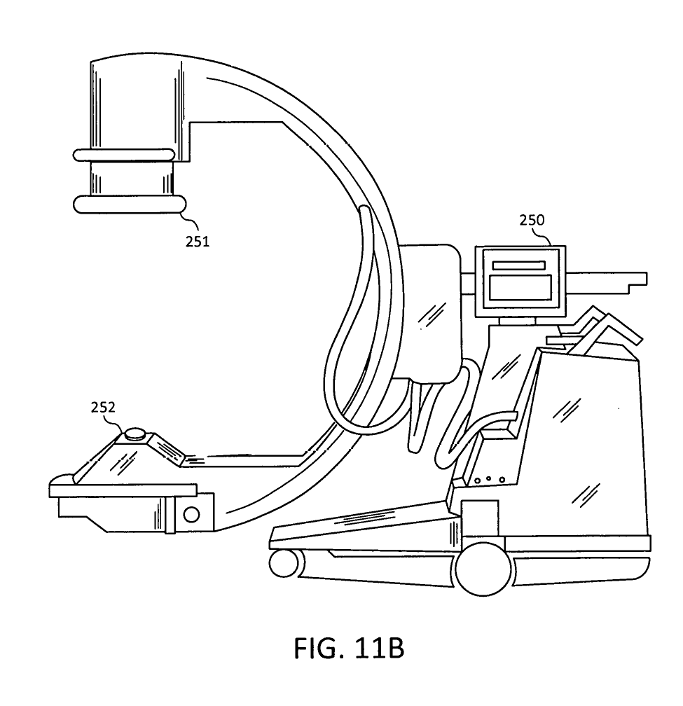

FIG. 11 A is a more detailed prospective view of a self-contained C-Arm Fluoroscopic system 200. The system 200 having an image intensifier 201, a grid 201, optics 203, a CCD camera 2014, monitors 205A and 205B, collimators 206, filters 207, X-ray tube 208, a generator 209 and automatic brightness control 210. The collar system 2 of FIG. 1 would fit around the circumference of the image intensifier 201 at 221 or around the Collimators 206 at 221. FIG. 11 B is a side view of a version 250 of the Smart Laser Aimer from GE OEC (GE Healthcare, Salt Lake City, Utah) noted earlier is one of the systems to be use with the invention where is shows the position of the collar system 2 in FIG. 1 can be placed at positions 251 and 252, depending on the position of the physician and the need entry point for surgery.

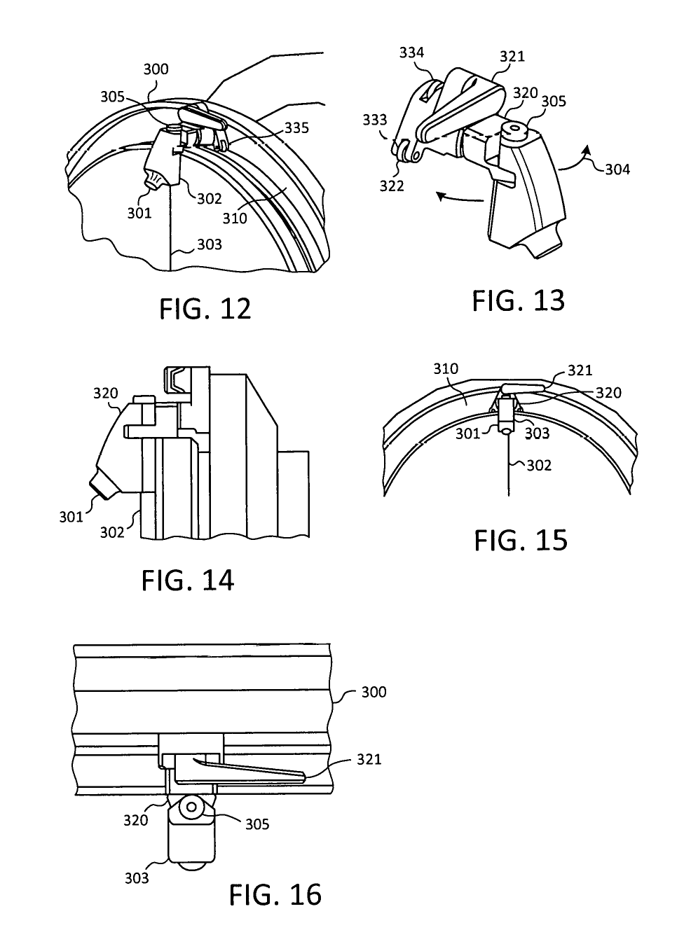

The collar system 2 discussed in FIG. 1 would fit around the circumference image intensifier 101 in FIG. 10 or the Collimator 110. FIG. 12 shows in a perspective cut away the collar system 300 with light source 301, which in this instance is a laser light source, a radiopaque marker 302 that is held in housing 303 and secured in the housing by fitting 305. The housing 303 is part of an assembly 320, shown more clearly in FIG. 13. FIG. 13 illustrates that the housing 303 has pivoting movement 304 in an arc of no more than plus or minus 5 degrees. Limiting the radiopaque bar/visible light marker to pivot on its axis to −+5° insures projected lines under fluoroscopy stay within the limits of beam divergence parameters for accuracy of visible light on patients skin. The radiopaque markers are always facing the center of the collar to minimize beam divergence. The entire assembly 320 fits into the circumferential channel 310 in FIG. 12. The entire assembly rotates around the circumference of the image intensifier of FIGS. 10 and 11 in the channel 310. The assembly has three wheels 333 and 334 in FIGS. 13 and 335 in FIG. 12 that permit circumferential movement around channel 310. When the proper location is found by viewing the radiopaque marker 302 as it appears on monitor 103 in FIG. 10 or Monitor 205 A.

The assembly 320 has a locking lever 321 that locks the assembly 320 in the desired circumferential position in channel 310 around the circumference of the image intensifier 101 in FIG. 10. The collar system 300 also fits around the circumference of the grid 202 and the assembly 320 would move around the circumference of the image intensifier 201 and the grid 201.

FIG. 14 a partial side view of the collar system with assembly 320 in channel 310 with the assembly having light source 310 and radiopaque 302 held in housing 303. FIG. 15 shows a partial front view of the collar system 300 showing channel 310, lock lever 321, housing 303 light source 301, and radiopaque marker 302. FIG. 16 shows a partial top view of collar system 300 and the assembly 320 with housing 303, fitting 305 and locking lever 321.

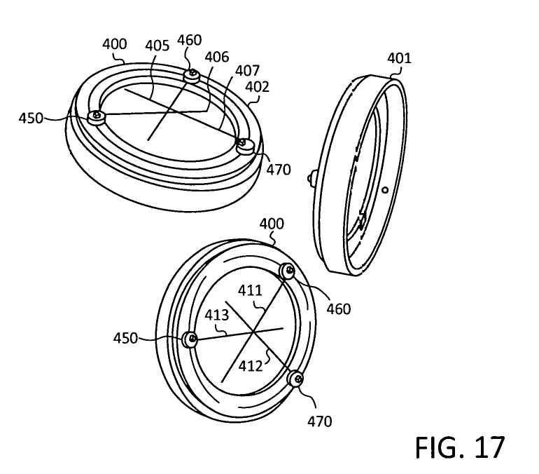

FIG. 17 illustrates a perspective view of collar system 400 having collar 401 that fits around the circumference of the image intensifier 101 or the Collimator 110 in FIG. 10 and around the circumference 202 at 220 or the Collimators 206 at 221 of FIG. 11. There is outer rim 402 where the assemblies 440, 450, and 460 rotate circumferentially around the grid 202 or the image intensifier 101 or the Collimator 110. There are also assemblies 450, 460, and 470 that have radiopaque markers 405, 406, and 407, as well as lights sources 411, 412, and 413, which illustrates that there can be alternative collar systems with multiple light sources and multiple radiopaque markers. Limiting the radiopaque bar/visible light marker to pivot on its axis to −+5° insures projected lines under fluoroscopy stay within the limits of beam divergence parameters for accuracy of visible light on patients skin. Also, the radiopaque markers are always facing the center of the collar to minimize beam divergence. With the introduction of square faces for the image intensifier or the collimator, the collar here can be easily constructed so that it was square to match up and have a circular channel and face to permitted the assemblies including the radiopaque markers and the light sources to travel around the circumference as shown. Further, with two assemblies on the collar system, the light sources can be arranged to create a target “x” by the intersection of the two light sources to create an entry point for medical instruments. Also, the two radiopaque marker may also be positioned to also permit a target “x” on that can be followed by the surgeon.

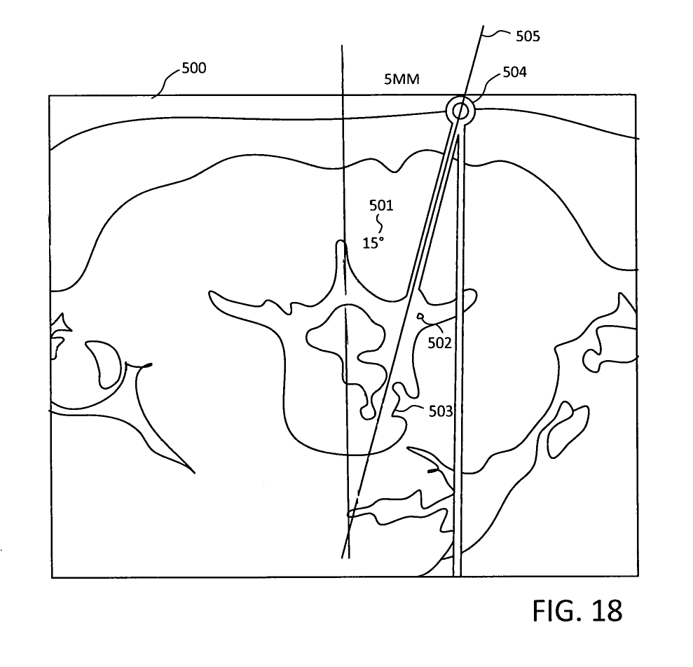

FIG. 18 illustrates the use of pre-surgical preparation where starting with a CT or MRI axial slice of the effected area, you can plot your angles such as the 15 degree angle 501 and identify landmarks such as 502 and 503 and where the skin port 504 for entry of the guide pin that will mimic the radiopaque position 505. This will normally be accomplished pre-operatively, but can also be accomplished intra-operatively as needed.

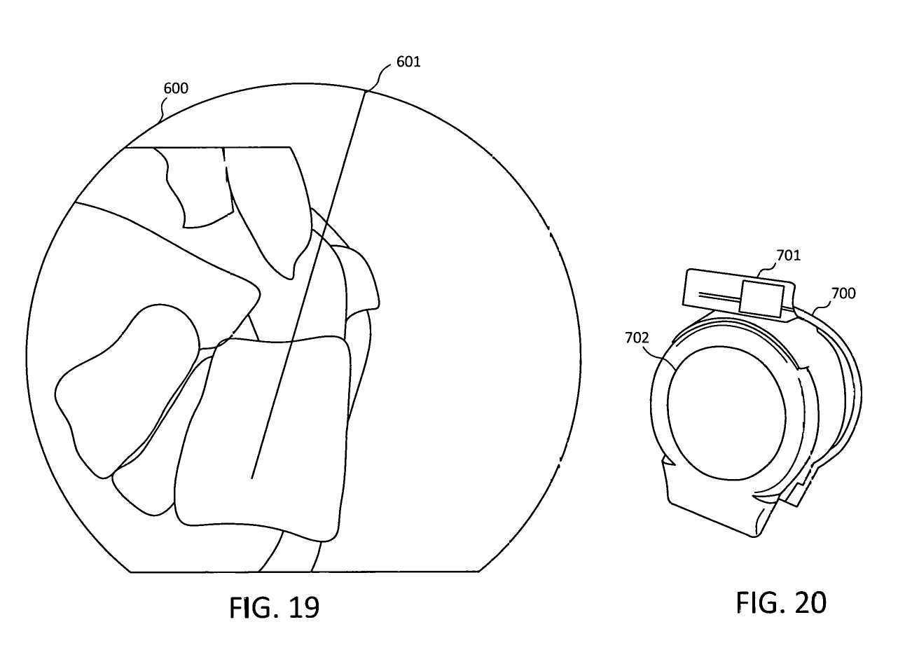

FIG. 19 shows a lateral image 600 having a radiopaque marker 601. The added accuracy is to have the guide pin insertion (not shown) to mimic or be position the same as the radiopaque marker 601 to provide a more accurate and quicker insertion by also using the AP angle or azimuth angle of 15 degrees. The radiopaque marker provides the surgeon with an insertion to replicate here for use in spine surgery or in any other type of surgery where precision and accuracy are required and the desire is to accomplish the same as minimally invasive.

FIG. 20 illustrates another version of a collar system for the fluoroscopic system 700, where the position of the radiopaque marker 702 is projected on image 600 which would be found on monitor 103 of Fluoroscopic system 100 in FIG. 10 or on monitor 205 A on fluoroscopic system in FIG. 11. The surgeon now has the image that she can precisely follow in inserting a guide pin.

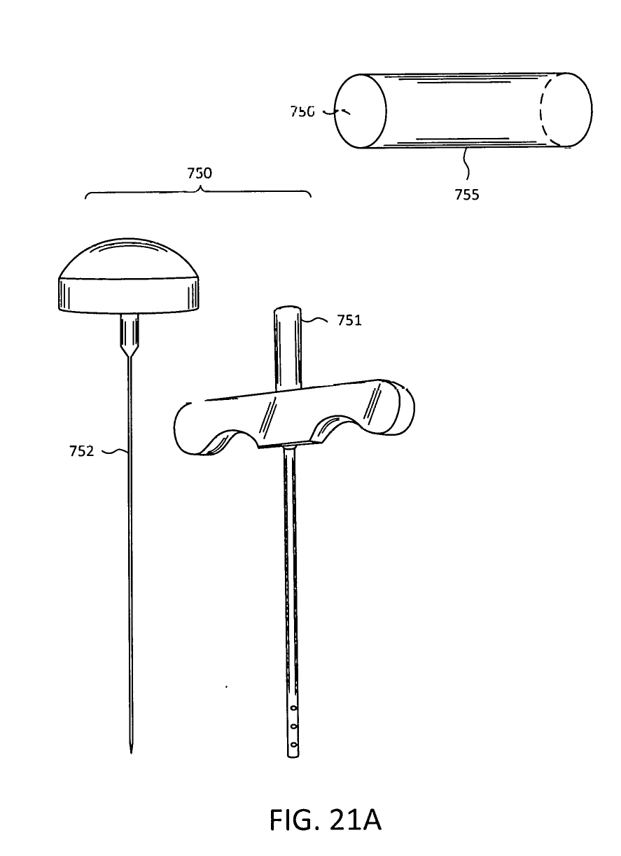

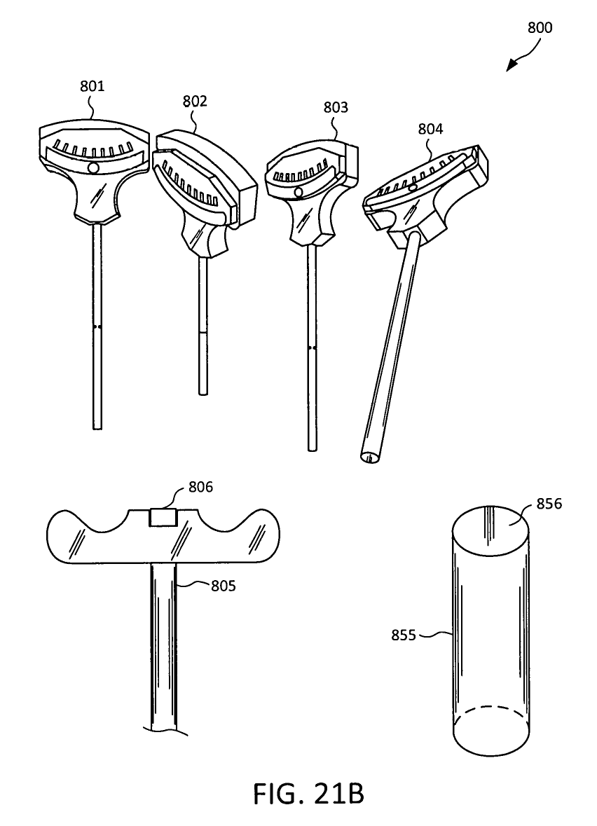

FIG. 21 A illustrates a Jamshidi 750 with stylet 752 and cannula 751 where the stylet slides into to complete the Jamshidi. Also, illustrated in FIG. 21 A is target guide holder 755 with an opening that goes completely through the center of 755. The target guide holder is made of a plastic that cannot be picked up by the X-rays of the fluoroscopic systems. The target guide 755 is held by a standard mechanical arm used in surgery so that it can be properly positioned by the position of the radiopaque marker and the angle in the AP Plane and the Angles in the ML plane for proper insertion of the surgical instruments. FIG. 21 B illustrates a series of bubble inclomaters 800 in various positions for inclometers 801, 802, 803, and 804. These inclometers will slide into the Jamshidi cannula 805 through opening 806 or the inclometers can slide in to the target guide holder 855 through opening 856 that goes through the entire length of the target guide holder 855 in order to determine the angel for the lateral plane and the AP plane for use correct angle and placement of the instruments such as a Jamshidi to make the initial incision.

FIG. 21 A illustrates a Jamshidi 750 that can be placed in a target guide holder 755. The holder would be held by a standard mechanical arm used in surgery (not shown) that would not be picked up on the x-ray of the fluoroscope system, whether they be system illustrated in FIG. 10, 11 A or 11 B or any other commercial fluoroscopic system. FIG. 21 B illustrates bubble inclomaters 800 that would be used to provide the correct angle of the Jamshidi. The stylet of Jamshidi 751 would be withdrawn and an inclomater such as 801 can be used by placing in the cannula of the Jamshidi 751 or 806 of 805 in FIG. 21B and the angle positioning can be determined for the AP Plane and the ML plane. Finally the position of the Jamshidi 750 in target guide holder 755 would be aligned with a mimic the position of the radiopaque marker 601 in FIG. 19. Then the surgeon can position the mechanical arm over the point of intersection of the entry point 51 of FIG. 4, which is where the two light sources intersect. Once there is the final position at point 51, the surgeon can then make the incision using the Jamshidi 750. The surgeon can also use a trocar, cannula, a drill bit or any surgical device used to make an incision at point 51 in FIG. 4. The instant invention and its many uses should not be limited to spine surgery, but can be used in surgery where there are two planes or even where there is a single plane of interest.

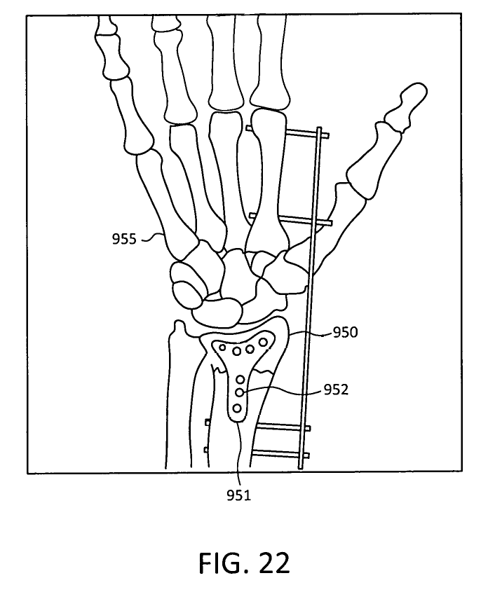

FIG. 22 is an illustration of a wrist 950 having a plate 951 and screws 952 with hand 955 that can benefit from the precision of the instant invention. Horizontal and vertical laser lines can be projected on the plane of the screw holes in the x and y plane and a radiopaque marker can be used to establish the correct position for inserting the screws 952.

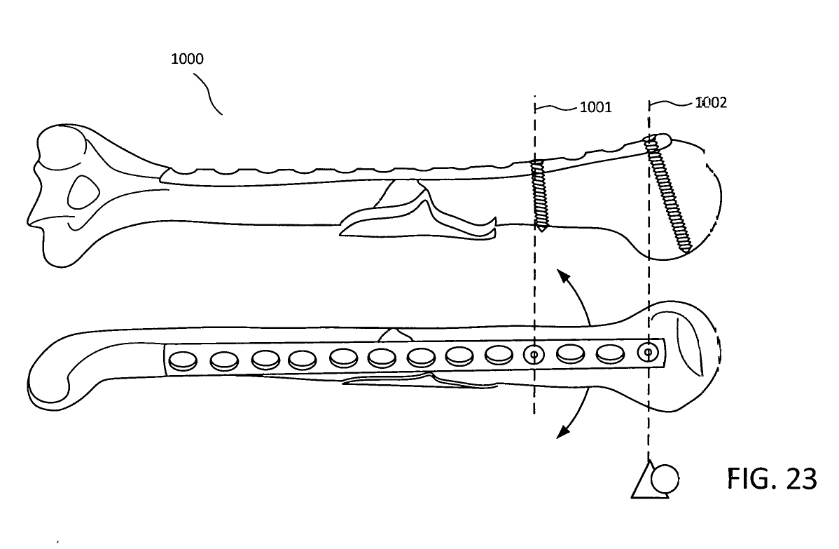

FIG. 23 is an illustration of a Humeral Shaft 1000 that can benefit from the instant invention as the screw lines 1001 and 1002 can be projected on the skin from the laser light sources, as well as the radiopaque marker, not shown, can illustrate further an exact duplication of the insert points for the screws.

The method and system here can be used not just for surgery but also for training of surgeons on cadavers or simulated bodies to improve technique and understanding. The training aspect of the instant invention is a key use of the method and system disclosed herein because it will provide a much more precise and accurate surgical technique being developed by surgeons.

Of course, the foregoing description is that of certain features, aspects and advantages of the present invention, to which various changes and modifications can be made without departing from the spirit and scope of the present invention.

Moreover, the surgical targeting systems and methods need not feature all of the objects, advantages, features and aspects discussed above. Thus, for example, those skilled in the art will recognize that the invention can be embodied or carried out in a manner that achieves or optimizes one advantage or a group of advantages as taught herein without necessarily achieving other objects or advantages as may be taught or suggested herein. In addition, while a number of variations of the invention have been shown and described in detail, other modifications and methods of use, which are within the scope of this invention, will be readily apparent to those of skill in the art based upon this disclosure. It is contemplated that various combinations or subcombinations of these specific features and aspects of embodiments may be made and still fall within the scope of the invention. Accordingly, it should be understood that various features and aspects of the disclosed embodiments can be combined with or substituted for one another in order to form varying modes of the discussed surgical marking systems and methods.

1. A method for surgery comprising:
(a) determining an anatomical landmark in the anterior-posterior (AP) plane;
(b) determining an angle for entry in the AP plane;
(c) determining an entry point in the (AP) plane;
(d) projecting a first light source orthogonal to the AP plane locating the anatomical landmark on the skin at a surgical site
(c) mounting a second light source on a radiographic imaging system projecting a light beam on the skin at the surgical site;
(d) mounting a radiopaque marker in alignment with the second light sourcing providing for said second light source projecting the position of the radiopaque marker through projection of its light beam in the ML plane;
(e) using the image of the radiopaque marker for the locating an anatomical location of interest using the radiographic imaging system;
(f) determining the angle of entry in the medial-lateral plane by referencing the imaged radiopaque marker; and
(g) locating the intersection of said first and said second light beams for marking the entry point incision for surgical instruments.

2. The invention of claim 1 where the angles of entry are determined by the use of inclomater.

3. The invention of claim 1 further comprising the step of having the surgical instrument making the initial incision instrument being held in place by a target guide holder.

4. The invention of claim 1 further comprising the step of aligning the surgical instrument in the target guide holder to be aligned with the position of the radiopaque marker.

5. The invention of claim 1 further comprising the step of have having said target guide holder be moveable in place by a mechanical arm.

6. The invention of claim 1 being used for training surgeons.

7. A method for surgery comprising;
(a) mounting a first light source on a radiographic imaging system projecting a first light beam on the skin at a surgical site;
(b) mounting a radiopaque marker in alignment with the first light sourcing providing for said light source projecting the position of the radiopaque marker image on the imaging system;
(c) positioning said radiopaque marker relative to the image on the imaging system of an anatomical landmark in ML plane;
(c) determining an anatomical landmark in the AP plane
(d) projecting a second light source light beam orthogonal to the anatomical landmark from the AP Plane on the skin of the surgical site; and
(e) locating the intersection of said first and said second light beams for marking the entry point for surgical instruments.

8. The invention of claim 7 comprising the further step of determining the angles of entry in the AP Plane and the ML Plane by use of an inclomater.

9. The invention of claim 7 where the angles of entry are determined by the use of inclomater.

10. The invention of claim 7 further comprising the step of having the surgical instrument making the initial incision instrument being held in place by a target guide holder.

11. The invention of claim 7 further comprising the step of aligning the surgical instrument in the target guide holder to be aligned with the position of the radiopaque marker.

12. The invention of claim 7 further comprising the step of have having said target guide holder be moveable in place by a mechanical arm.

13. The invention of claim 7 used for training surgeons.

14. A method for surgery comprising:
(a) determining an anatomical landmark in the anterior-posterior (AP) plane;
(b) determining an angle for entry in the AP plane;
(c) determining an entry point in the (AP) plane;
(d) using a marking pen for making a line orthogonal to the AP plane locating the anatomical landmark on the skin at a surgical site
(e) mounting a light source on a radiographic imaging system projecting a light beam on the skin at the surgical site;
(f) positioning said radiopaque marker relative to the image on the imaging system of an anatomical landmark
(g) mounting a radiopaque marker in alignment with the second light sourcing providing for said light source projecting the position of the radiopaque marker through projection of its light beam;
(h) imaging the radiopaque marker for with the locating anatomical location of interest using imaging of the radiographic imaging system in the ML plane;
(i) determining the angle of entry in the medial-lateral plane by referencing the imaged radiopaque marker; and
(j) locating the intersection of said marking pen line with said light beam for marking the entry point for surgical instruments.

15. The invention of claim 14 further comprising the step of having the surgical instrument making the initial incision instrument being held in place by a target guide holder.

16. The invention of claim 14 further comprising the step of aligning the surgical instrument in the target guide holder to be aligned with the position of the radiopaque marker.

17. The invention of claim 14 further comprising the step of have having said target guide holder be moveable in place by a mechanical arm.

18. The invention of claim 14 used for training surgeons.

19. A system for surgery comprising;
(a) an imaging system;
(b) an radio plaque marker;
(c) a light source;
(d) a mechanical arm;
(e) an inclomater;
(f) a target guide holder; and
(g) a surgical instrument.

20. The invention of claim 19, where the imaging system can be portable.
